(19) United States
(12) Patent Application Publication
(10) Pub. No.: US 2004/0081746 A1
(43) Pub. Date: Apr. 29, 2004

(54) Method for regenerating container for plasma treatment, member inside container for plasma treatment, method for preparing member inside container for plasma treatment, and apparatus for plasma treatment

(76) Inventor: Kosuke Imafuku, Yamanashi (JP)

Correspondence Address: Finnegan Henderson Farabow, Garrett & Dunner, 1300 I Street NW, Washington, DC, 20005, US

(21) Appl. No.: 10/450,094

(22) PCT Filed: Dec. 7, 2001
(86) PCT No.: PCT/JP01/10715

(30) Foreign Application Priority Data
Dec. 12, 2000 (JP) 2000-377100

(51) Int. Cl. A61L 2/00; B05D 3/00

(52) U.S. Cl. 427/2.3

(57) ABSTRACT

When a sprayed coating, which is constituted of alumina, a rare earth oxide, polyimide or polybenzimidazole and covers a surface of a base material of a plasma processing container internal member, becomes degraded through use in a plasma environment, the same material as that constituting the sprayed coating is resprayed over the degraded sprayed coating. As a result, the plasma processing container with its surface having become degraded through use in the plasma environment can be restored to an as-good-as-new state.

### BACKGROUND OF THE INVENTION

The present invention relates to a method for recycling a plasma processing container, a plasma processing container internal member, a method for manufacturing a plasma processing container internal member and a plasma processing apparatus, and more specifically, it relates to a method for recycling a plasma processing container through which a member with a surface thereof having become degraded through use in a plasma environment can be recycled to an as-good-as-new state.

A plasma processing apparatus such as an etching apparatus is normally employed during the process of manufacturing devices constituted of semiconductors, liquid crystal or the like. Since a reactive gas such as CF₄ is used as the process gas inside such a plasma processing apparatus (inside the plasma processing container), the internal members tend to become chemically damaged and they are also prone to sustain erosion damage due to ions and the like having become excited by the plasma.

For this reason, plasma processing container internal members are protected in the related art by covering the surfaces of their base materials such as an aluminum material with a film that does not readily become worn away by plasma. A sprayed coating constituted of alumina, a rare earth oxide or the like, which is not readily worn off by plasma in particular, is often used for this purpose in the related art. Alternatively, plasma processing container internal members constituted of a material such as aluminum are protected by setting polyimide coating having a thickness of, for instance, 1.5 mm over their bases.

In this type of plasma processing apparatus, numerous replaceable components (hereafter referred to as apparatus components) achieving electrical conductivity or an insulating property, such as a focus ring, a shield ring and the like, are provided at specific positions in the processing chamber.

In the plasma processing apparatus described above, as the surfaces of the apparatus components become ground and deformed by plasma generated inside the processing chamber, the deformed portions are disposed of as expendable items and replaced with new components.

Since it is inevitable that degradation of a sprayed coating starts to occur at its surface after use over an extended period of time to result in reduced film thickness, the service life of the internal member covered with this sprayed coating is determined by the extent to which the film thickness has decreased. Once its service life has expired, it must be replaced with a new member, which is not cost effective. In addition, since the surface of the sprayed coating is highly irregular and particles of the reaction product resulting from the reaction with the process gas are readily formed, particularly at projections on the sprayed coating surface, during the initial stage of the plasma processing container internal member utilization. Such particles may cause a defect in the product.

Likewise, it is necessary to replace a coating constituted of polyimide or the like once its surface becomes degraded. In addition, a gap is bound to be created between the base and the resin layer. If the contact between the base and the resin layer is poor., problems including a buildup of dirt, are bound to occur.

Furthermore, when a replaceable apparatus component becomes worn and deformed, the deformed portion is disposed of as an expendable item and is replaced with a new component, as described above. However, it is bound to be costly to frequently replace a worn apparatus component with a new component, and there is also a problem in that if no replacement is in stock, the production line has to be stopped.

An object of the present invention, which has been completed by addressing the problems of plasma processing container internal members in the related art discussed above, is to provide a new and improved method for recycling a plasma processing container, a new and improved plasma processing container internal member, a new and improved method for manufacturing a plasma processing container and a new and improved plasma processing apparatus.

Another object of the present invention is to provide a method for recycling a plasma processing container through which a substitute is prepared by repairing an apparatus component that has become partially deformed through a simple repair process.

### SUMMARY OF THE INVENTION

In order to achieve the objects described above, a first aspect of the present invention provides a method for recycling a plasma processing container characterized in that as a sprayed coating constituted of alumina, a rare earth oxide, polyimide or polybenzimidazole, which covers the surface of the base material of an internal member provided inside a plasma processing container, has become degraded through use in a plasma environment, a material identical to that constituting the sprayed coating is sprayed again over the degraded sprayed coating. By adopting this method, it becomes possible to recycle the plasma processing container with its surfaces having become degraded through use in a plasma environment to an as-good-as-new state.

In the method for recycling a plasma processing container described above, a step during which dry ice is blasted may be executed before the material identical to that constituting the sprayed coating is sprayed. By executing such a step, the extent of particle generation at the early stage can be reduced. In addition, a dry ice blast may be executed after coating is re-sprayed.

In order to achieve the objects described above, a second aspect of the present invention provides a method for recycling a plasma processing container characterized in that as a component provided at a specific position inside the plasma processing container becomes partially deformed during plasma processing, the deformed portion is first removed and then a part formed to achieve the pre-deformation shape is bonded onto the area from which the deformed portion has been removed. In this case, since only the deformed portion of the apparatus component having become partially deformed needs to be replaced with a part formed to achieve the pre-deformation shape, the apparatus component can be restored to the original shape through a simple repair process without having to replace the entire apparatus component with a new component.

In order to achieve the objects described above, a third aspect of the present invention provides a plasma processing container internal member having a surface of the base material of thereof covered with a sprayed coating constituted of alumina, are rare earth oxide, polyimide or polybenzimidazole characterized in that dry ice is blasted onto the film after it is sprayed.

In order to achieve the objects described above, a fourth aspect of the present invention provides a method for manufacturing a plasma processing container internal member, comprising a step in which the surface of the base material of the internal member is coated with a sprayed coating constituted of alumina, a rare earth oxide, polyimide or polybenzimidazole and a step in which dry ice is blasted onto the film having been sprayed.

According to the present invention achieved in the third and fourth aspects, the extent of particle generation at the early stage is minimized. In addition, it becomes possible to provide a plasma processing container internal member that can be restored to an as-good-as-new state without any functional degradation after a subsequent sprayed coating by minimizing the extent of particle generation at the early stage of its use and a method for manufacturing such a plasma processing container internal member

### BRIEF DESCRIPTION OF THE DRAWINGS

FIG. 1 shows the structure adopted in a plasma processing apparatus in the first and second embodiments;

FIG. 2 presents schematic sectional views of the plasma processing container internal member achieved in the first embodiment;

FIG. 3 presents schematic sectional views illustrating the process through which the plasma processing container internal member is recycled in the first embodiment;

FIG. 4 presents schematic sectional views illustrating the process through which the plasma processing container internal member is recycled in the second embodiment;

FIG. 5 shows the internal structure adopted in the etching apparatus constituting the plasma apparatus in the third embodiment;

FIG. 6 is a sectional view of the focus ring;

FIG. 7 illustrates a mode of implementation of the method for recycling the plasma apparatus component achieved in the third embodiment;

FIG. 8 is a sectional view of the shield ring and

FIG. 9 illustrates another mode of implementation of the method for repairing the plasma apparatus component achieved in the third embodiment.

### DETAILED DESCRIPTION OF THE PREFERRED EMBODIMENTS

The following is a detailed explanation of the preferred embodiments of the method for recycling a recyclable plasma processing container, the plasma processing container internal member, the method for manufacturing the plasma processing container internal member and the plasma processing apparatus according to the present invention.

The recyclable plasma processing container internal member according to the present invention may be any of the various members provided inside the plasma processing apparatus, including a deposit shield, a baffle plate, a focus ring, an insulator ring, a shield ring, a bellows cover and an electrode. The explanation below mainly focuses on an example in which the present invention is adopted in a semiconductor manufacturing apparatus.

(First and Second Embodiments)

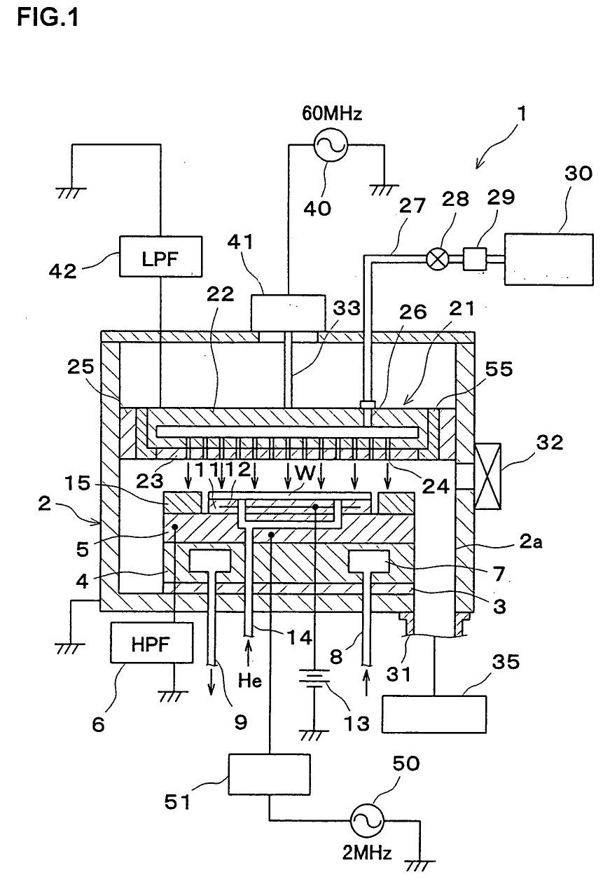

FIG. 1 is a sectional view of the structure of a plasma apparatus 1 achieved in the first and second embodiments of the present invention. A processing chamber 2 of the plasma apparatus 1 is formed as a cylindrical processing container constituted of a base material which may be, for instance, aluminum having undergone an anodic oxide coating process. The processing chamber 2 is grounded.

An insulating support plate 3, which may be constituted of ceramic, is provided at the bottom inside the processing chamber 2, and a susceptor support stage 4 formed in a substantially columnar shape, on which a substrate (e.g., a semiconductor wafer W with an 8-inch diameter) is placed, is provided above the insulating support plate 3. A susceptor 5 constituting a lower electrode is provided on top of the susceptor support stage 4, with a high pass filter (HPF) 6 connected to the susceptor 5.

A heat exchanging chamber 7 is provided inside the susceptor support stage 4. A heat exchanging medium flows in from the outside to circulate inside the heat exchanging chamber 7 via a heat exchanging medium supply pipe 8 and a heat exchanging medium discharge pipe 9 so as to sustain the temperature of the semiconductor wafer W at a predetermined level via the susceptor 5. In addition, automatic control is implemented by using a temperature sensor (not shown) and a temperature control mechanism (not shown) to sustain the temperature of the semiconductor wafer at the predetermined level.

An electrostatic chuck 11 that attracts and holds the semiconductor wafer W is provided on top of the susceptor 5. The electrostatic chuck 11 adopts a structure achieved by clamping, for instance, an electrically conductive thin film electrode 12 with a polyimide resin from the top and the bottom, and as a voltage of, for instance, 1.5 kV is applied to the electrode 12 from a D.C. source 13 provided outside the processing chamber 2, the wafer W is held firmly onto the upper surface of the electrostatic chuck 11 due to the resulting coulomb force. However, it goes without saying that the wafer W may be held onto the susceptor 5 without utilizing such an electrostatic chuck but instead by using a mechanical clamp to press the edges of the wafer W onto the susceptor 5.

A gas passage 14 through which an He gas or the like is supplied to the rear surface of the semiconductor wafer W is formed at the insulating plate 3, the susceptor support stage 4, the susceptor 5 and the electrostatic chuck 11, and the temperature of the semiconductor wafer W is sustained at the predetermined level with the He gas which is used as a heating medium.

Over the upper edge of the susceptor 5, a substantially annular-shaped focus ring 15 is provided so as to enclose the electrostatic chuck 11. The focus ring 15, which may be constituted of, for instance, an electrically conductive silicon, has a function of allowing ions in the plasma to enter the semiconductor wafer W efficiently.

Toward the top inside the processing chamber 2, an upper electrode 21 is supported via an insulating member 25 and a shield ring 55. The upper electrode 21 includes an electrode support member 22 constituted of, for instance, aluminum, an electrode plate 23 constituted of silicon or the like which is set in parallel to the susceptor 5 so as to face opposite the susceptor 5 and includes numerous outlet holes 24. The susceptor 5 and the upper electrode 21 are separated from each other by, for instance, approximately 10 m m-60 mm.

A gas supply port 26 is formed at the electrode support member 22, and the gas supply port 26 is connected to a gas supply pipe 27. Through the gas supply pipe 27 connected to a process gas supply source 30 via a valve 28 and a mass flow controller 29, an etching gas and another process gas are supplied into the processing chamber 2 from the process gas supply source 30.

The process gas may be a gas containing a halogen element, such as fluorocarbon gas (CₓF_{y}) or hydrofluorocarbon gas (CₚH_{q}Fᵣ).

At the bottom of the processing chamber 2, an evacuation pipe 21 linked to an evacuation device 35 constituted of a vacuum pump and the like is connected. The evacuation device 35 having a vacuum pump which may be a turbo molecular pump is capable of evacuating the processing chamber 2 to achieve a desired lowered pressure level, e.g., 10 mTorr-1000 mTorr.

A gate valve 32 is provided at the side wall of the processing chamber 2, and the semiconductor wafer W is transferred between the processing chamber and the adjacent load lock chamber (not shown) while the gate valve 32 is set in an open state.

Next, the high-frequency power supply system of the plasma apparatus 1 is explained. Power from a first high-frequency source 40 which outputs high-frequency power achieving a frequency of, for instance, 27 MHz-150 MHz is supplied to the upper electrode 21 via a matcher 41 and a feeding rod 33. In addition, a low pass filter (LPF) 42 is connected to the upper electrode 21.

By applying high frequency power, as described above, a high-density plasma achieving a desirable state of dissociation can be formed inside the processing chamber 2 to enable plasma processing under low pressure conditions. The high-frequency source 4 may be capable of outputting power at, for instance, 60 MHz.

Power from a high-frequency source 50 that outputs high-frequency power with a frequency of 4 MHz or lower, for instance, is supplied to the susceptor 5 constituting the lower electrode via a matcher 51. By applying power at a frequency in such a range, desirable ionization can be achieved without damaging the semiconductor wafer W.

The plasma processing container internal member achieved in the embodiment, which is exposed to the plasma during the processing in the plasma processing apparatus 1 described above, may be an inner wall 2a of the processing chamber 2, the insulating support plate 3, the susceptor support stage 4, the susceptor 5, the electrostatic chuck 11, the focus ring 15, the insulating member 25 or the shield ring 55.

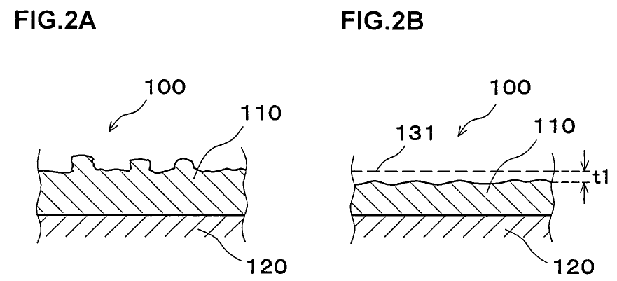

FIG. 2 presents schematic sectional views of the plasma processing container internal member 100 achieved in the embodiment. FIG. 2A shows the plasma processing container internal member 100 immediately after spraying the sprayed coating and FIG. 2B shows the plasma processing container internal member 100 after a CO₂ blast. As shown in FIG. 2A, a sprayed coating 110 is formed at the surface of a base material 120 of the plasma processing container internal member constituted of, for instance, Al. The sprayed coating 110 may be constituted of alumina (Al₂O₃), a rare earth oxide, polyimide or polybenzimidazole.

In the related art, a resin such as polyimide is used to protect the base material by, for instance, setting a polyimide coating with a 1.5 mm thickness over the Al base material, and the resin is replaced as the polyimide resin layer becomes degraded through use in the plasma environment.

While sprayed coating is achieved through the collision impact attributable to the heat and the eruption speed in the related art, sprayed coating is achieved in the embodiment through the collision impact attributable to the eruption speed alone. As a result, it is possible to spray coat a film having a film thickness of approximately several millimeters and a film thus formed constitutes the sprayed coating.

While an Al₂O₃ sprayed coating or a Y₂O₃ sprayed coating should be ideally formed through the atmospheric plasma spraying method or through plasma spraying executed within an atmosphere that substantially does not contain any oxygen, a sprayed coating may instead be formed through high-speed flame spraying or detonation flame spraying.

The surface of the film formed through any of the methods described above is in a highly irregular state immediately after the sprayed coating, and if the film is used inside the plasma processing container in this state, particles are generated readily, particularly at projecting, fragmented layers (cracked layers) due to collisions with ions from the plasma to cause degradation of the film.

Accordingly, CO₂ is blasted onto the film immediately after the sprayed coating process shown in FIG. 2B to flatten out the irregularities at the surface of the film. As a result, a state similar to the condition of the plasma processing container internal member having been used inside the plasma processing container over a specific length of time is achieved for the newly sprayed film, which makes it possible to minimize the extent of particle generation at an early stage. It is to be noted that the sprayed coating surface 131 in FIG. 2A becomes ground over a thickness of t1 through the CO₂ blast process.

The CO₂ blast should be executed under conditions that include, for instance, the pressure set to 2.5 kgf/cm²-4.2 kgf/cm², the nozzle diameter set to 16 mm, the distance between the nozzle and the sprayed coating surface set to 15 mm, the particle diameter of the dry ice set to 0.3 mm-20 mm and the dry ice rate set to 0.5 kg/min. When the sprayed coating is constituted of Y₂O₃, for instance, it is desirable to ensure that the extent t1 to which the film thickness becomes reduced through the CO₂ blast does not exceed 10 μm.

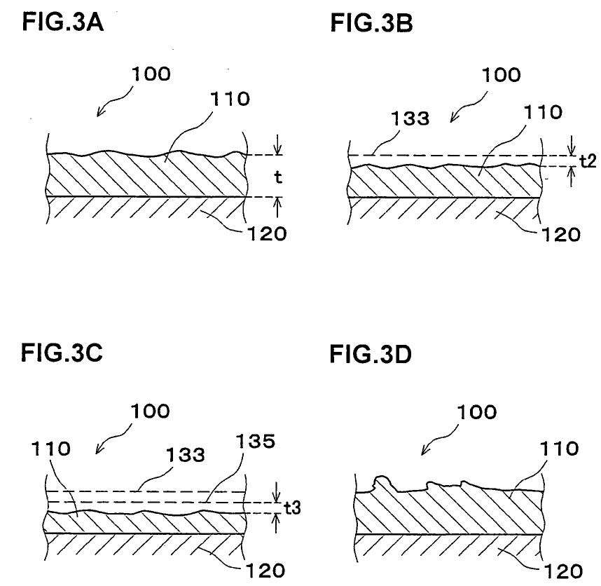

FIG. 3 presents schematic sectional views of the process through which the plasma processing container internal member 100 is recycled in the first embodiment. FIG. 3A shows the plasma processing container internal member 100 at the initial state (the plasma processing container internal member 100 has been blasted with CO₂ but has not been used in the plasma processing container yet), whereas FIG. 3B shows the plasma processing container internal member 100 after it has been used in the plasma processing container. FIG. 3C shows the plasma processing container internal member 100 after a CO₂ blast executed for purposes of renewing and FIG. 3D shows the plasma processing container internal member 100 after the respray. The term “respraying” as used in this context refers to an act of sprayed coating more film over the sprayed coating formed before the plasma processing, after the plasma processing container internal member 100 has been used inside the plasma processing container.

FIG. 3A shows the plasma processing container internal member 100 with the sprayed coating 110 formed at the surface of the base material 120 constituted of, for instance, Al and the surface of the sprayed coating 110 flattened through a CO₂ blast. The sprayed coating 110 may be constituted of alumina, a rare earth oxide, polyimide or polybenzimidazole.

Y₂O₃, which is a rare earth oxide, should be sprayed to achieve a thickness t of 50 μm-2000 μm, whereas polyimide or polybenzimidazole should be sprayed to achieve a thickness t of, for instance, 2 mm-3 mm. These are values considered appropriate for achieving both the damage preventing effect and an acceptable level of cost performance. When such a sprayed coating 110 is exposed to the plasma, the sprayed coating surface 133 becomes worn by a thickness t2 relative to the surface level 133 in FIG. 3A, as shown in FIG. 3B.

Table 1 below lists the varying extents t2 to which the film thicknesses of different types of sprayed coatings covering the plasma processing container internal member became reduced after having been left in a plasma processing apparatus. It is to be noted that the plasma processing container internal member was left for 20 hours in a plane parallel plasma etching apparatus with the chamber pressure set to 40 mTorr, the R F power level set to 1500 W and the etching gas was a mixed gas containing CF₄, Ar and O₂ at a ratio of 100:20:200.

TABLE 1

| | |
|---|---|
| 1) | film type |
| 2) | extent of wear t2 (μm) |
| 3) | Y₂O₃ sprayed coating |
| 4) | Al₂O₃ sprayed coating |
| 5) | Al₂O₃ ceramic |

As Table 1 indicates, good plasma erosion resistance was demonstrated by Y₂O₃ and Al₂O₃ even in an atmosphere containing halogen compounds. Under the conditions described above, the Y₂O₃ sprayed coating among the four different types of films became worn off the least, demonstrating outstanding plasma resistance.

Next, the CO₂ blast executed on the Y₂O₃ sprayed coating is explained. The CO₂ blast was executed under conditions which include the pressure set to 2.5 kgf/cm²-4.2 kgf/cm², the nozzle diameter set to 16 mm, the distance between the nozzle and the sprayed surface set to 15 mm, the particle diameter of the dry ice set to 0.3 mm-2.0 mm and the dry ice rate set to 0.5 kg/min.

When the blast was executed over durations of 30 sec and 60 sec, blast quantities of 5 μm and 10 μm were achieved respectively. Through this process, the sprayed coating surface became ground over a thickness of t3 relative to the surface level 135 in FIG. 3B, as shown in FIG. 3C and, as a result, the irregular surface was flattened and foreign matter was removed. It is to be noted that in the case of the Y₂O₃ sprayed coating, the extent t3 to which the film thickness becomes reduced through the CO₂ blast should be preferably 10 μm or more and more desirably, 20 μm or more.

Next, as shown in FIG. 3D, a material identical to that constituting the sprayed coating 110 is resprayed. Since no crystal change occurs within the film constituted of alumina, a rare earth oxide, polyimide or polybenzimidazole over time, new crystals are formed continuously to the old crystals at the interface during the respray and thus, the sprayed coating is restored to an as-good-as-new state. In addition, a CO₂ blast may be executed again subsequently to flatten the irregular surface of the sprayed coating.

As explained above, by adopting the recyclable plasma processing container internal member which minimizes the extent of particle generation at the initial stage, the method for manufacturing this plasma processing container internal member and the method for recycling the plasma processing container internal member achieved in the first embodiment of the present invention, a plasma processing container internal member that does not allow particles to be generated readily at the initial stage and can be restored to an as-good-as-new state after use can be provided.

In addition, while it is desirable to remove the surface of the plasma processing container internal member after use through a CO₂ blast that does not allow any foreign matter to remain at the surface, the surface may be cleaned through a method other than a CO₂ blast. As long as the surface can be washed with a chemical solution or the like to achieve a clean state without damaging the sprayed coating or the base material, a blast may be executed by using alumina or SiC, or the surface may be polished through sanding with abrasive grains or the like. Alternatively, the surface may be chemically polished through an etching process executed by using a chemical solution.

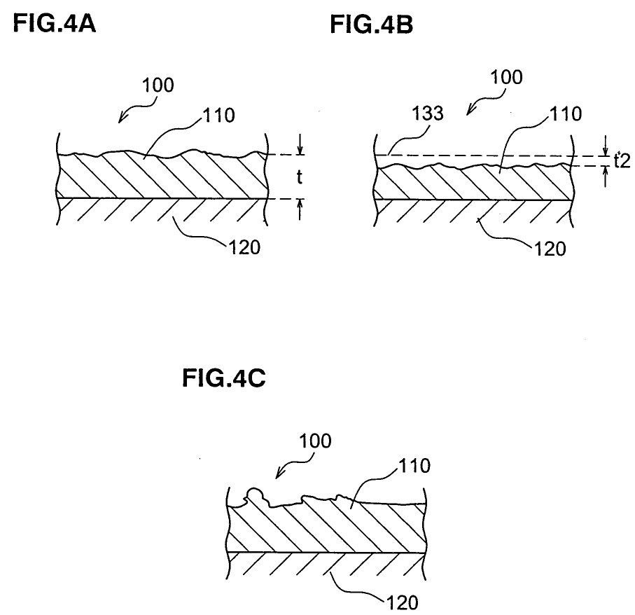

Now, the plasma processing container internal member achieved in the second embodiment is explained in reference to the drawings. FIG. 4 presents schematic sectional views of the process during which the plasma processing container internal member 100 is recycled in the second embodiment. FIG. 4A shows the plasma processing container internal member 100 in the initial state, and FIG. 4B shows the plasma processing container internal member 100 after it has been used inside the plasma processing container. FIG. 4C shows the plasma processing container internal member 100 after the respray.

Unlike in the first embodiment, no CO₂ blast is executed after the plasma processing container internal member has been used inside the plasma processing container but instead, a respray is executed by using the same material as that constituting the sprayed coating after the sprayed coating has been left during use inside the plasma processing container in the second embodiment. The conditions adopted in the second embodiment are identical to those adopted in the first embodiment, except that no CO₂ blast is executed in the second embodiment.

There is an advantage to executing a respray by using the same material as that constituting the pre-plasma processing sprayed coating without executing a CO₂ blast in that the sprayed coating deposited through the respray is allowed to set more easily, since the spray coated film deposited through the respray is allowed to adhere more readily onto the irregular surface with projections and indentations after the plasma processing than onto a relatively flat surface. As a result, the plasma processing container with its surface having become degraded through use in a plasma environment can be recycled to an as-good-as-new state.

(Third Embodiment)

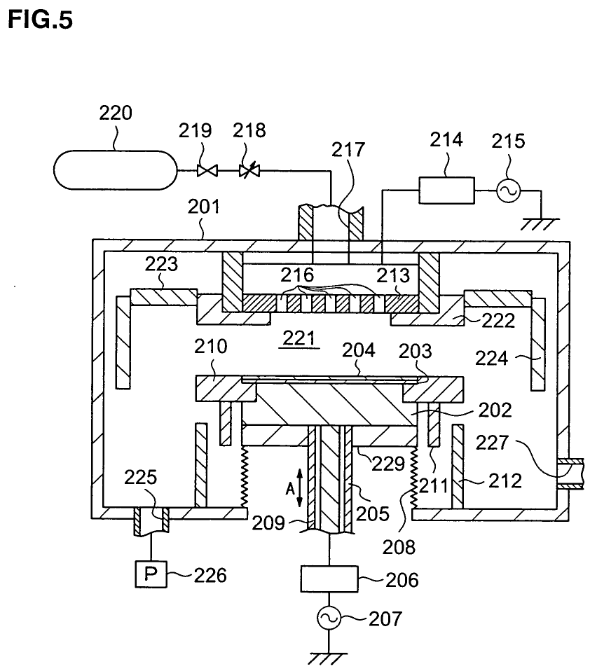

Next, the third embodiment of the present invention is explained in reference to the drawings. FIG. 5 shows the internal structure of a plasma etching apparatus constituting the plasma processing apparatus. Inside an apparatus main unit 201 of this plasma etching apparatus, i.e., inside a processing chamber 221, numerous types of apparatus components each formed in a specific shape are provided at specific positions.

More specifically, a lower electrode 202 constituted of an electrically conductive material is provided toward the bottom of the processing chamber 221, and an electrostatic chuck 204 which holds a semiconductor wafer W, i.e., the substrate, is provided on the lower electrode 202. The lower electrode 202 is supported by an elevator shaft 205 capable of moving upward/downward along the directions indicated by the arrow A. The elevator shaft 205 is connected to a high-frequency source 207 via a matcher 206. The elevator shaft 205 passes through an annular member 209 constituted of an electrically conductive material.

In addition, the lower electrode 202 is protected by an electrode holding member 229. Between the electrode support member 229 and the bottom surface of the apparatus main unit 201, an expandable bellows 208 constituted of an electrically conductive material such as stainless steel is provided. A focus ring 210 constituted of an electrically conductive material or an insulating material is provided at the upper portion of the side surface of the lower electrode 202, with a first bellows cover 211 provided at the bottom surface of the focus ring 210. From the bottom surface of the apparatus main unit 201, a second bellows cover 211 rises so as to partially overlap the first bellows cover 211.

Toward the top of the processing chamber 221, an upper electrode 213 constituted of an electrically conductive material is provided so as to face opposite to the lower electrode 202, and the upper electrode 213 is connected to a high-frequency source 215 via a matcher 214. Numerous gas outlet holes 216 are formed at the upper electrode 213. At the top surface of the apparatus main unit 201, a gas supply port 217 is provided, and a reactive gas containing a CF (fluorocarbon) gas is supplied into the processing chamber 221 through the gas outlet holes 216 through the gas supply port 217. More specifically, the gas supply port 217 is connected to a gas supply source 220 via a flow regulating valve 218 and a switching valve 219, and thus, the reactive gas from the gas supply source 220 is supplied to the gas supply port 217 via the switching valve 219 and the flow-regulating valve 218, and is then let into the processing chamber 221 through the gas outlet holes 216.

In addition, the upper electrode 213 is held by a shield ring 222 formed by using an insulating material. A protective ring 223 is provided so as to enclose the periphery of the shield ring 222. From the external circumferential edge of the protective ring 223, a shield member 224 extends vertically along the downward direction.

At the bottom of the apparatus main unit 201, a discharge hole 225 is provided. The discharge hole 225 is connected to a vacuum pump 226. At the side surface of the apparatus main unit 201 toward the bottom, a substrate transfer hole 227 is provided. The semiconductor wafer W is carried into the processing chamber 22 land also is carried out from the processing chamber 221 via the substrate transfer hole 227.

In the plasma etching apparatus structured as described above, the elevator shaft 205 is caused to move along the directions indicated by the arrow A by a drive mechanism (not shown) to adjust the position of the semiconductor wafer W. Then, as high-frequency power with a frequency of, for instance, 13.56 MHz is applied to the lower electrode 202 and the upper electrode 213 from the high-frequency source 207 via the elevator shaft 205 which functions as a feeding rod and from the high-frequency source 215, a glow discharge occurs.

As the pressure inside the processing chamber 221 is reduced by the vacuum pump 226 to achieve a specific vacuum atmosphere and the reactive gas from the gas supply source 220 is supplied into the processing chamber 221, plasma is generated from the reactive gas through the glow discharge, the plasma is trapped in the space between the lower electrode 202 and the upper electrode 213 by the focus ring 210 and the shield ring 222 and, as a result, a desired type of fine processing is executed on the semiconductor wafer W having specific masking thereupon. Thus, the semiconductor wafers W becomes finely processed through dry etching.

During this process, the surfaces of the various apparatus components that are exposed to the plasma environment, such as the focus ring 210 and the shield ring 222, also become etched and worn and thus, these worn apparatus components need to be replaced with new components depending upon the extent of the wear.

However, if such worn apparatus components are constantly replaced with new components, the production cost is bound to rise, and if no replacements are in stock, the production line has to be stopped.

Accordingly, in the third embodiment, if a given component becomes partially deformed, the deformed portion is removed and a part formed to achieve the original shape is welded and bond onto the area where the deformed portion has been removed.

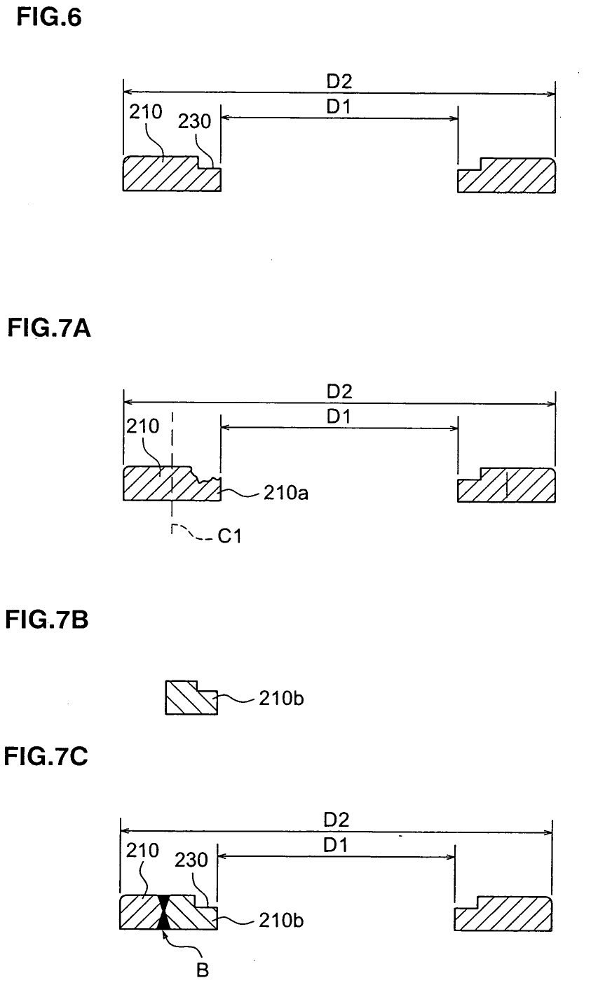

FIG. 6 shows a sectional view of the focus ring 210. Under normal circumstances, a new focus ring 210 is formed in a ring shape with an internal diameters D1 and an external diameter D2, which includes a stage portion 330 at its internal circumferential surface.

The focus ring 210, which is constituted of an electrically conductive material such as Al or an insulating material such SiO₂, improves the consistency of the plasma around the semiconductor wafer W when constituted of an electrically conductive material and raises the density of the plasma formed over the semiconductor wafer W when constituted of an insulating material. Since the focus ring 210 is exposed to the plasma atmosphere in either case, its surface is etched with the plasma and becomes ground off. As a result, the focus ring 210 becomes partially deformed with a deformed portion 210a emerging over the area.

Accordingly, a new part 210b achieving the original dimensions and shape is separately produced, as shown in FIG. 7B, the deformed portion 210a is removed by cutting the focus ring 210 along a cutline C1 in FIG. 7A, and the new part 210b is welded and bonded onto the area (the area B in FIG. 7C) corresponding to the deformed portion 210a, thereby producing a focus ring 210 having a stage portion 230 identical to that shown in FIG. 6 at its internal circumferential surface in the embodiment. The focus ring 210 having been restored to the original state through this repair is set at the specific position at the plasma etching apparatus and the desired type of etching processing is resumed subsequently.

As described above, even if the focus ring 210 becomes partially deformed through etching, the focus ring 210 can be restored to the desired state simply by removing the deformed portion 210a and replacing it with the new part 210b in the third embodiment. As a result, it becomes no longer necessary to replace the entire focus ring having become deformed with a new focus ring every time, and the apparatus component repaired through a simple process can be utilized as an acceptable substitute, which leads to a reduction in production costs.

In addition, the present invention as achieved in the third embodiment may likewise be adopted in other apparatus components provided at the plasma etching apparatus, including the shield ring 222, the protective ring 223 and the shield member 224.

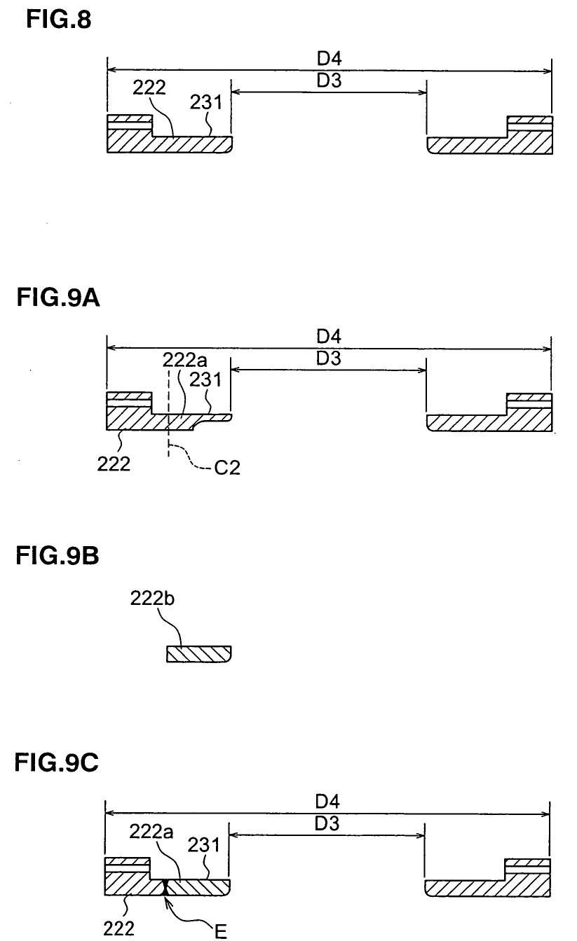

FIGS. 8 and 9 illustrate how the method for plasma apparatus component recycling achieved in the third embodiment may be adopted to restore the shield ring 222.

FIG. 8 is a sectional view of the shield ring 222. Under normal circumstances, a new shield ring 222 is formed in a ring shape having an internal diameter D3 and an external diameter D4 and includes a thin walled portion 231.

Since the shield ring 222, too, is exposed to the plasma as is the focus ring 210, the thin walled portion 231 becomes partially etched and a deformed portion 222a emerges over time, as shown in FIG. 9.

Accordingly, a new part 222b achieving the original dimensions and shape is separately produced, as shown in FIG. 9B, the deformed portion 222a is removed by cutting the shield ring 222 along a cutline C2 in FIG. 9A, and the new part 222b is welded and bonded onto the area (eg., the area E in FIG. 9C) corresponding to the deformed portion 222a, thereby producing a shield ring 222 having a thin walled portion 231 identical to that shown in FIG. 8 in the embodiment, as in the case of the focus ring 210 (see FIG. 7). The shield ring 222 having been restored to the original state through this repair is set at the specific position at the plasma etching apparatus and the desired type of etching processing is resumed subsequently.

As described above, even if the shield ring 222 becomes partially deformed through etching, the shield ring 222 can be restored to the desired state simply by removing the deformed portion 222a and replacing it with the new part 222b, as in the case of the focus ring 210. As a result, it becomes no longer necessary to replace the entire focus ring having become deformed with a new focus ring each time, and the apparatus component repaired through a simple process can be utilized as an acceptable substitute, which leads to a reduction in production costs.

It is to be noted that while an explanation is given above in reference to the third embodiment on an example in which the present invention is adopted in a so-called ion-assist-type plasma etching apparatus, the present invention is not limited to this example and may be instead adopted in, for instance, a magnetic field assist-type plasma etching apparatus.

As explained above, according to the present invention, in which the same material as that constituting the sprayed coating such as alumina, a rare earth oxide, polyimide or polybenzimidazole, covering the surface of the base material of a plasma processing container internal member is resprayed as the surface of the sprayed coating becomes degraded through use in a plasma environment, the plasma processing container with its surface having become degraded through use in a plasma environment can be restored to an as-good-as-new state.

In addition, if a component disposed at a specific position inside the plasma processing container becomes partially deformed during the plasma processing, the deformed portion is first removed and then a part achieving the original shape is bonded onto the area from which the deformed portion has been removed. As a result, it is no longer necessary to replace the entire apparatus component having become partially deformed with a new apparatus component every time and instead, the apparatus component can be restored as an acceptable substitute through a simple process, to achieve a reduction in production costs. Furthermore, even when a specific spare component is out of stock, the down time of the production line can be minimized.

Moreover, the extent of particle generation at the initial stage can be minimized by covering the surface of the base material of a plasma processing container internal member with a sprayed coating constituted of alumina, a rare earth oxide, polyimide or polybenzimidazole and by smoothing the surface of the sprayed coating through a CO₂ blast prior to use.

While the invention has been particularly shown and described with respect to preferred embodiments of the method for recycling a plasma processing container internal member and the recyclable plasma processing container internal member by referring to the attached drawings, the present invention is not limited to these examples and it will be understood by those skilled in the art that various changes in form and detail may be made therein without departing from the spirit, scope and teaching of the invention.

The present invention may be adopted to restore a plasma processing container internal member with its surface having become degraded through use in a plasma environment into an as-good-as-new state and more specifically, it may be effectively adopted in the process of manufacturing semiconductor devices, LCD substrates and the like.

What is claimed is:

1. A method for recycling a plasma processing container, wherein:
when a sprayed coating, which is constituted of alumina, a rare earth oxide, polyimide or polybenzimidazole and covers a surface of a base material of an internal member of said plasma processing container, becomes degraded through use in a plasma environment, a material identical to the material constituting said sprayed coating is resprayed over said sprayed coating.

2. A method for recycling a plasma processing container according to claim 1, wherein:
a dry ice blast process is executed before the material is resprayed.

3. A method for recycling a plasma processing container according to claim 1 or 2, wherein:
a dry ice blast process is executed after the material is resprayed.

4. A method for recycling a plasma processing container used to execute plasma processing on a substrate placed therein, wherein:
if a component provided at a specific position inside said plasma processing container becomes partially deformed during said plasma processing, the deformed portion is first removed and then a part achieving the original shape prior to the deformation is bonded onto the area from which said deformed portion has been removed.

5. A plasma processing container internal member having a surface of a base material thereof covered with a sprayed coating constituted of alumina, a rare earth oxide, polyimide or polybenzimidazole, with said sprayed coating, which has been sprayed onto said surface, blasted with dry ice.

6. A method for manufacturing a plasma processing container internal member, comprising:
a step in which a surface of a base material is covered with a sprayed coating constituted of alumina, a rare earth oxide, polyimide or polybenzimidazole; and
a step in which said sprayed coating having been sprayed onto said surface is blasted with dry ice.

7. A plasma processing container internal member having a surface of a base material thereof covered with a sprayed coating constituted of polyimide or polybenzimidazole.

8. A plasma processing apparatus that executes plasma processing by adopting a method for recycling a plasma processing container according to any of claims 1 through 4.

9. A plasma processing apparatus having a plasma processing container internal member according to claim 5.
